(19) United States
(12) Patent Application Publication
(10) Pub. No.: US 2019/0313983 A1
(43) Pub. Date: Oct. 17, 2019

(54) USER TERMINAL APPARATUS

(71) Applicants: OMRON CORPORATION, Kyoto-shi (JP); OMRON HEALTHCARE CO., LTD., Kyoto (JP)

(72) Inventors: Natsuko HORIGUCHI, Kyoto-shi (JP); Hiroshi NAKAJIMA, Kyoto-shi (JP); Tomohiro KUKITA, Kyoto (JP); Hirotaka WADA, Kyoto-shi (JP); Tamio UEDA, Osaka (JP)

(21) Appl. No.: 16/454,331

(22) Filed: Jun. 27, 2019

Related U.S. Application Data
(63) Continuation of application No. PCT/JP2017/044395, filed on Dec. 11, 2017.

(30) Foreign Application Priority Data
Jan. 4, 2017 (JP) 2017-000246

(51) Int. Cl. A61B 5/00; A61B 5/021; A61B 5/024; A61B 5/0255

(52) U.S. Cl. CPC A61B 5/7475; A61B 5/021; A61B 5/02405; A61B 5/0002; A61B 5/7282; A61B 5/742; A61B 5/0255; A61B 5/486

(57) ABSTRACT

According to one embodiment, a user terminal apparatus includes a hardware processor and a memory connected to the hardware processor. The hardware processor is configured to detect whether a user has entered a specific state, acquire a heart rate of the user which is measured by a sensor, determine whether the measured heart rate is less than a threshold when it is detected that the user has entered the specific state, and issue a notification to prompt to take an action when it is determined that the measured heart rate is less than the threshold.

### CROSS-REFERENCE TO RELATED APPLICATIONS

This application is a Continuation Application of PCT Application No. PCT/JP2017/044395, filed Dec. 11, 2017 and based upon and claiming the benefit of priority from Japanese Patent Application No. 2017-000246, filed Jan. 4, 2017, the entire contents of all of which are incorporated herein by reference.

### FIELD

Embodiments of the present invention relate to a user terminal apparatus that measures biological information of a user.

### BACKGROUND

Measurement results of the biological information of a user are used in various scenes such as health care. For example, Jpn. Pat. Appln. KOKAI Publication No. 2013-128748 discloses a technique of measuring the heart rate of a user and issuing a notification when the measured heart rate is not a normal value.

In recent years, with advances in sensor technology, for example, a user terminal apparatus has been implemented which can measure the biological information of a user by being worn on a wrist of the user. This user terminal apparatus can measure the biological information of a user without applying much burden on the user.

For example, there is a medicine that effectively works when the user takes or is administered it at a specific timing such as an empty stomach time. It is difficult for the user to take such a medicine at a proper timing. In addition, the user sometimes forgets to take the medicine. A user terminal apparatus as described above is required to be able to issue a notification to prompt the user to take an action such as taking or being administered a medicine based on a measurement result of the biological information of the user.

### SUMMARY

A first aspect of the present invention is a user terminal apparatus including a detection unit configured to detect whether a user has entered a specific state, a sensor configured to measure a heart rate of the user or measure a blood pressure waveform of the user to calculate a heart rate from a period of the measured blood pressure waveform, a determination unit configured to determine whether the heart rate is less than a threshold when it is detected that the user has entered the specific state, and a notification unit configured to issue a notification to prompt to take an action when it is determined that the heart rate is less than the threshold.

According to the first aspect, the heart rate of a user is measured, and a desired timing to take a predetermined action is detected based on the measured heart rate, thereby issuing a notification to prompt the action. This allows the user to take an action at a proper timing or another person to take an action for the user at a proper timing.

In a second aspect of the present invention, the action is to take or be administered a medicine.

According to the second aspect, the user can take a medicine at a proper timing or another person such as a doctor can administer a medicine to the user at a proper timing.

In a third aspect of the present invention, the sensor is a blood pressure sensor configured to measure a blood pressure value per heartbeat based on the measured blood pressure waveform.

According to the third aspect, a heart rate can be measured by using a blood pressure sensor.

In a fourth aspect of the present invention, the user terminal apparatus further includes an input unit configured to receive a user input indicating that the user has entered the specific state, wherein the detection unit performs detection based on the user input.

According to the fourth aspect, it is indicated that the user has entered a specific state. This makes it possible to easily detect that the user has entered the specific state.

In a fifth aspect of the present invention, the user terminal apparatus further includes a learning unit configured to perform learning to identify that the user has entered the specific state based on sensor data obtained by the sensor, wherein the detection unit detects, based on sensor data obtained by the sensor, whether the user has entered the specific state.

According to the fifth embodiment, it is possible to automatically detect that the user has entered the specific state.

### BRIEF DESCRIPTION OF THE DRAWINGS

FIG. 1 is a block diagram showing a user terminal apparatus according to an embodiment of the present invention;

FIG. 2 is a perspective view showing an example of the outer appearance of the user terminal apparatus shown in FIG. 1;

FIG. 3 is a view showing an example of the arrangement of a biological information management system including the user terminal apparatus shown in FIG. 1;

FIG. 4 is a graph showing the relationship between heart rate and medication timing; and

FIG. 5 is a flowchart showing an example of a procedure in which the user terminal apparatus shown in FIG. 1 notifies a medication timing.

### DETAILED DESCRIPTION

According to an embodiment of the present invention, there is provided a user terminal apparatus that can issue a notification to prompt the user to take an action based on a measurement result of the biological information of the user.

Hereinafter, embodiments of the present invention will be described with reference to the accompanying drawings.

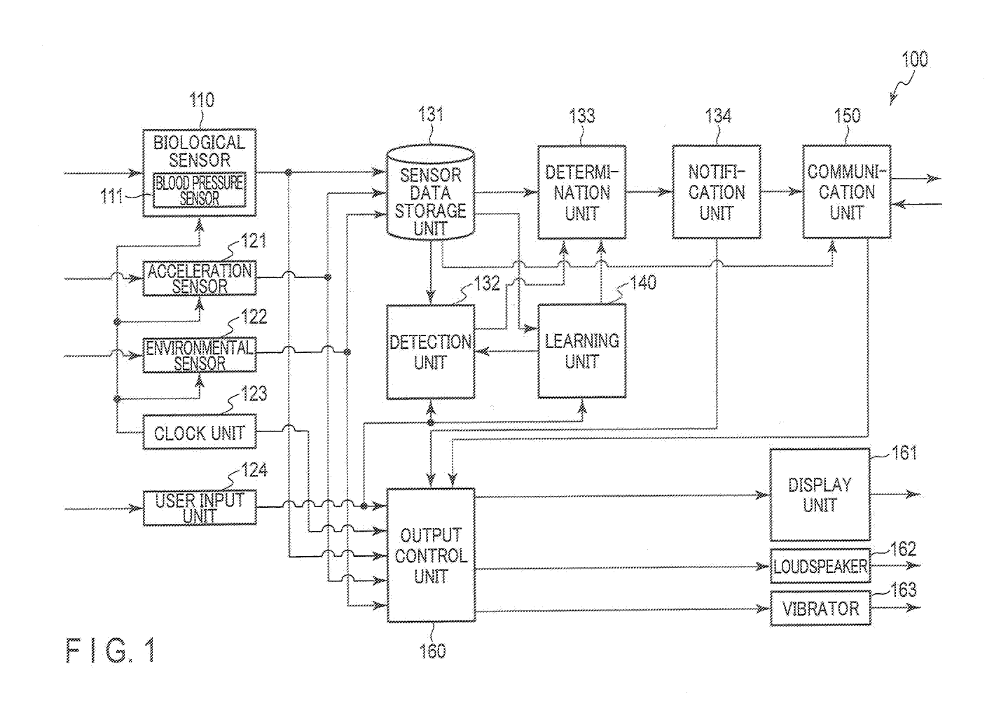

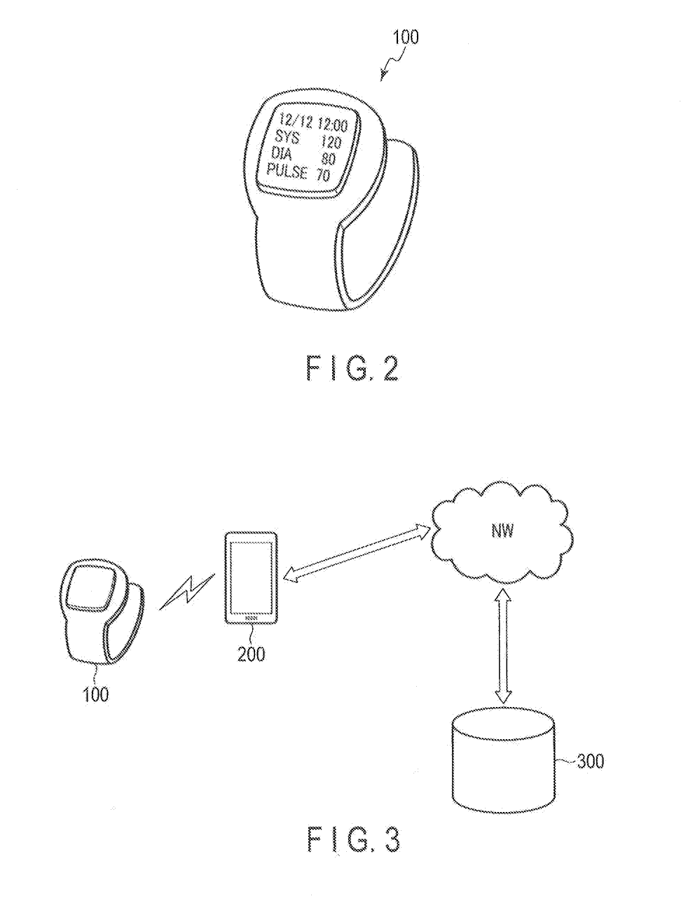

FIG. 1 schematically shows a user terminal apparatus 100 according to one embodiment of the present invention. The user terminal apparatus 100 shown in FIG. 1 may be a wearable device such as a wristwatch-type wearable device illustrated in FIG. 2. The user terminal apparatus 100 can measure biological information such as the systolic blood pressure (SBP), diastolic blood pressure (DBP), heart rate, and the like of the user who wears the user terminal apparatus 100. A heart rate indicates the number of heartbeats per unit time (for example, 1 min). The user terminal apparatus 100 can display a measurement result together with information displayed on a general clock, such as a date and time.

As shown in FIG. 3, the user terminal apparatus 100 may be connected to a smart device 200. Typically, the smart device 200 may be a portable device such as a smartphone or tablet. The smart device 200 can display biological data transmitted by the user terminal apparatus 100 in a graphical form and transmit the biological data to a server 300 via a network NW. An application that manages biological data may be installed in the smart device 200. Note that the user terminal apparatus 100 may be connected to the server 300 via the network NW without via the smart device 200.

The server 300 stores biological data transmitted from the user terminal apparatus 100 or the smart device 200. The server 300 may transmit the biological data in response with an access made from a personal computer (PC) installed in a medical institution to use the biological data, for example, for medical care guidance or diagnosis of the user.

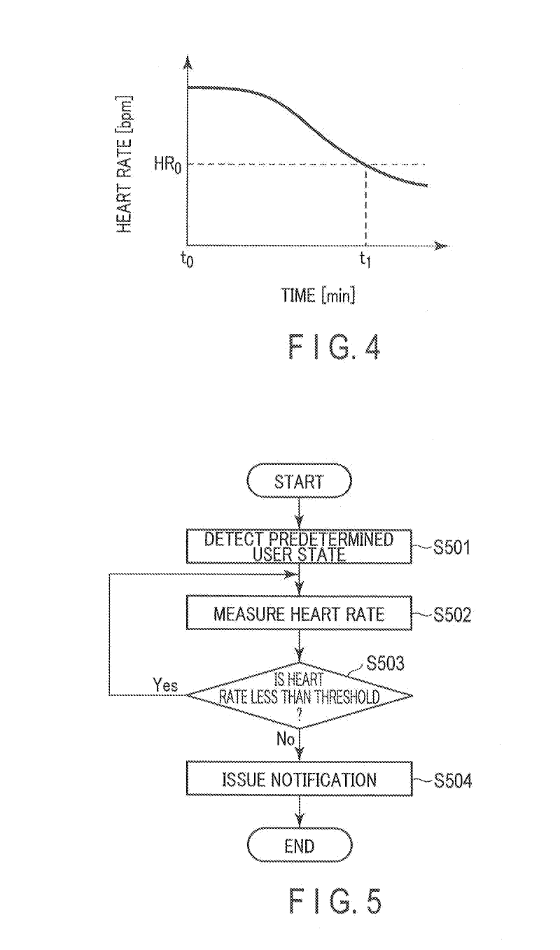

The user terminal apparatus 100 issues a notification to prompt the user or an associated person to take an action. Assume that a medicine to be taken by the user at empty stomach after eating is prescribed. In this case, the user terminal apparatus 100 determines whether the user has become empty stomach after eating, and issues a notification to prompt the user to take a medicine in accordance with determination that the user has become empty stomach. This processing will be described in detail below with reference to FIG. 4.

FIG. 4 shows variations in heart rate after eating. Referring to FIG. 4, HR₀ represents a threshold concerning heart rates, to represents the end time of eating, and t₁ represents the time when the heart rate becomes the threshold HR₀. It is known that the heart rate at full stomach is higher than that at empty stomach. As shown in FIG. 4, when the user takes a meal, the heart rate temporarily becomes high and gradually decreases with the lapse of time. For example, when the heart rate becomes less than the threshold HR₀, the user terminal apparatus 100 can determine that the user has become hungry. In this case, the threshold HR₀ is set to a value that makes it possible to regard that the user has become hungry. The threshold HR₀ can be set depending on the user.

The user terminal apparatus 100 measures the heart rate of the user by using a biological sensor (to be described later). When the heart rate after eating becomes less than the threshold HR₀, the user terminal apparatus 100 issues a notification to prompt the user to take a medicine. This enables the user to take the medicine at a proper timing. In addition, this makes it possible to prevent the user from forgetting to take the medicine. It is possible to detect, based on the sensor data output from the biological sensor, that the user has taken a meal. For example, the detection can be performed based on at least one of electrocardiogram data, heart rate data, pulse wave data, pulsation data, and body temperature data. Alternatively, the detection may be performed based on a user input. Note that when, for example, a doctor is to administer a medicine to the user, the user terminal apparatus 100 may transmit a notification to prompt the user to take the medicine to an external device.

Referring to FIG. 1, the user terminal apparatus 100 includes a biological sensor 110, an acceleration sensor 121, an environmental sensor 122, a clock unit 123, a user input unit 124, a sensor data storage unit 131, a detection unit 132, a determination unit 133, a notification unit 134, a communication unit 150, an output control unit 160, a display unit 161, a loudspeaker 162, and a vibrator 163.

The biological sensor 110 measure the biological information of the user to obtain biological data, and sends the biological data to the sensor data storage unit 131 and the output control unit 160. The biological sensor 110 includes, as an example, a blood pressure sensor 111 that measures the blood pressure of the user to obtain blood pressure data. In this case, biological data includes blood pressure data. In addition, the biological data can include electrocardiogram data, heart rate data, pulse wave data, pulsation data, and body temperature data. Each biological data can be associated with measurement time set based on the time information received from the clock unit 123.

The blood pressure sensor 111 includes a continuous blood pressure sensor. The continuous blood pressure sensor is a blood pressure sensor capable of continuously measuring blood pressures per heartbeat (for example, a systolic blood pressure and a diastolic blood pressure). The continuous blood pressure sensor can be based on a technique of measuring a pulse transmit time (PTT) and estimating a blood pressure from the measured pulse transmit time, a tonometry method, or another technique. The blood pressure data obtained by the continuous blood pressure sensor includes, but is not limited to, blood pressure values per heart beat (for example, a systolic blood pressure and a diastolic blood pressure). The continuous blood pressure sensor can measure the blood pressure waveform of the user and obtain blood pressure values based on the measured blood pressure waveform. The continuous blood pressure sensor then can calculate a heart rate based on the period of the measured blood pressure waveform. The heart rate data includes, but is not limited to, a heart rate. A heart rate is not limited to that measured by the continuous blood pressure sensor, and may be measured by a heart rate sensor.

The blood pressure sensor 111 may further include a noncontinuous blood pressure sensor. A noncontinuous blood pressure sensor includes, as an example, a blood pressure sensor based on an oscillometric method of measuring a blood pressure by using cuff as a pressure sensor. The noncontinuous blood pressure sensor (an oscillometric blood pressure sensor in particular) tends to have higher measurement accuracy than a continuous blood pressure sensor. Accordingly, the blood pressure sensor 111 is triggered by, for example, satisfying a certain condition (for example, when the blood pressure data of the user measured by the continuous measurement type blood pressure sensor indicates a predetermined high risk state) to operate the noncontinuous blood pressure sensor in place of the continuous blood pressure sensor, thereby measuring blood pressure data with high accuracy.

The acceleration sensor 121 detects the acceleration applied to the acceleration sensor 121 to obtain three-axis acceleration data. This acceleration data can be used to estimate the activity state (posture and/or action) of the user wearing the user terminal apparatus 100. The acceleration sensor 121 sends the acceleration data to the sensor data storage unit 131 and the output control unit 160. The acceleration data can be associated with the measurement time set based on the time information received from the clock unit 123.

The environmental sensor 122 measures environmental information around the user terminal apparatus 100 to obtain environmental data, and sends the environmental data to the sensor data storage unit 131 and the output control unit 160. The environmental data can include temperature data, humidity data, and atmospheric pressure data. Each environmental data can be associated with the measurement time set based on the time information received from the clock unit 123.

The clock unit 123 generates time information representing the current time in a predetermined cycle, and sends the time information to the biological sensor 110, the acceleration sensor 121, the environmental sensor 122, and the output control unit 160. Time information can be used as the time of measurement of biological data by the biological sensor 110, the time of measurement of acceleration data by the acceleration sensor 121, and the time of measurement of environmental data by the environmental sensor 122. The clock unit 123 may include a calendar function. That is, the clock unit 123 may generate date information representing the current date and send the date information to the output control unit 160.

The user input unit 124 includes, for example, buttons and dials for the reception of user inputs. Alternatively, a combination of the user input unit 124 and the display unit 161 (to be described later) may be implemented by using a touch screen. A user input is, for example, the operation of inputting a user state such as eating and awaking, the operation of controlling the display screen of the display unit 161, or the like. A user input corresponding to eating may be performed at the start of eating, at the end of eating, and during eating.

The sensor data storage unit 131 stores the biological data output from the biological sensor 110, the acceleration data output from the acceleration sensor 121, and the environmental data output from the environmental sensor 122.

The detection unit 132 detects whether the user has entered a specific state. The detection unit 132 can detect that the user has entered a specific state, based on a user input from the user input unit 124. For example, when the user operates the user input unit 124 so as to indicate that the user has taken a meal, the detection unit 132 detects that the user has taken a meal. Alternatively, the detection unit 132 may detect, based on sensor data from the sensor data storage unit 131, whether the user has entered a specific state. The detection unit 132 may detect, based on information from an external device obtained through the communication unit 150, that the user has entered a specific state. Upon detecting that the user has entered a specific state, the detection unit 132 sends a detection signal to the determination unit 133.

In response to the reception of a detection signal from the detection unit 132, the determination unit 133 determines, based on sensor data from the sensor data storage unit 131, whether a predetermined condition is satisfied. Referring to the example described with reference to FIG. 4, the determination unit 133 receives heart rate data from the sensor data storage unit 131, and compares the heart rate with the threshold HR₀ to determine that the condition is satisfied, when the heart rate becomes less than the threshold HR₀. The determination unit 133 sends information indicating that the predetermined condition is satisfied to the notification unit 134.

It is also known that eating will increase the blood pressure value. In another embodiment, the determination unit 133 may determine that the condition is satisfied, when the blood pressure value (for example, the systolic blood pressure) becomes less than another threshold. In still another embodiment, the determination unit 133 may determine that the condition is satisfied, when the heart rate becomes less than the threshold HR₀ and the blood pressure value becomes less than another threshold. Using both a heart rate and a blood pressure value makes it possible to perform determination more accurately.

Upon receiving information from the determination unit 133, the notification unit 134 issues a notification to prompt an action. The notification unit 134 can send a notification to the output control unit 160. Alternatively, the notification unit 134 may send a notification to prompt an action to the communication unit 150 upon receiving information from the determination unit 133.

The communication unit 150 exchanges data with an external device such as the smart device 200 or the server 300 shown in FIG. 2. The communication unit 150 performs one or both of wireless communication and wired communication. For example, the communication unit 150 performs short range wireless communication such as Bluetooth® with the smart device 200. Upon receiving a notification from the notification unit 134, the communication unit 150 can transmit data including the notification to an external device. In addition, the communication unit 150 can receive sensor data from the sensor data storage unit 131 and transmit data including the sensor data. The communication unit 150 can also receive data from an external device. For example, the data received from an external device is sent to the output control unit 160 and output to the user.

The output control unit 160 controls an output interface. In this embodiment, the output interface includes the display unit 161, the loudspeaker 162, and the vibrator 163. The output control unit 160 can generate screen data and send it to the display unit 161. For example, the output control unit 160 generates screen data based on biological data from the biological sensor 110, acceleration data from the acceleration sensor 121, environmental data from environmental sensor 122, time information and date information from the clock unit 123, a notification from the notification unit 134, or data from the communication unit 150. For example, upon receiving a notification from the notification unit 134, the output control unit 160 generates screen data including a message for prompting an action such as “take the medicine”. The output control unit 160 can generate an acoustic signal and send it to the loudspeaker 162. For example, the output control unit 160 generates an acoustic signal based on a notification from the notification unit 134. The output control unit 160 generates a voltage signal for vibrating the vibrator 163 and applies the voltage signal to the vibrator 163. For example, the output control unit 160 generates a voltage signal based on a notification from the notification unit 134.

The display unit 161 is, for example, a liquid crystal display or organic electroluminescence (EL) display. The display unit 161 can notify the user of various information by displaying screen data from the output control unit 160. More specifically, the display unit 161 may display information based on information from the notification unit 134, biological information (for example, a blood pressure, electrocardiogram, heart rate, pulse wave, pulse rate, and body temperature), acceleration data, activity amount information (for example, the number of steps counted based on acceleration data and a consumed calorie), sleep information (for example, a sleep time), environmental information (for example, a temperature, humidity, and atmospheric pressure), the current time, a calendar, and information from an external device. The loudspeaker 162 converts an acoustic signal from the output control unit 160 into an acoustic wave. The vibrator 163 vibrates in accordance with a voltage signal from the output control unit 160.

The user terminal apparatus 100 may further include a learning unit 140. The learning unit 140 can perform supervised learning for identifying, based on sensor data, that the user has entered a specific state. For example, the learning unit 140 uses, as correct answer data, the sensor data obtained at the time of reception of a user input indicating the ingestion of a meal or in a time zone before the ingestion of a meal. It is known that the ingestion of a meal exerts influence on the circulatory system. For this reason, for example, heart rate data, blood pressure data, or their combination can be used as sensor data to be used to detect whether the user has entered a specific state. The detection unit 132 receives the learning result obtained by the learning unit 140. This allows the detection unit 132 to detect whether the user has entered the specific state by using the sensor data acquired from the sensor data storage unit 131. In addition, the learning unit 140 may determine a threshold (for example, the threshold HR₀) set in the determination unit 133 based on the sensor data acquired from the sensor data storage unit 131 and a user input.

The user terminal apparatus 100 includes, for example, a central processing unit (CPU) and a memory as hardware. The memory includes a read only memory (ROM), random access memory (RAM), and secondary storage device. Various types of functions of the user terminal apparatus 100 can be implemented by causing the CPU to read out a program from the ROM or secondary storage device into the RAM and executing the program. As the secondary storage device, for example, a semiconductor memory or hard disk drive (HDD) can be used. The secondary storage device includes the sensor data storage unit 131. Note that some or all of the functions of the user terminal apparatus 100 may be implemented by hardware such as IC (integrated circuit) chips.

An operation of the user terminal apparatus 100 will be described next.

FIG. 5 shows an example of the operation of the user terminal apparatus 100. In step S501 in FIG. 5, the detection unit 132 detects that the user has entered a predetermined state. Subsequently, in step S502, the determination unit 133 acquires the heart rate data of the user measured by the blood pressure sensor 111. In step S503, the determination unit 133 determines whether the heart rate has become less than threshold. If the heart rate is equal to or more than the threshold, the process returns to step S502. If the heart rate is less than the threshold, the process advances to step S504. In step S504, the notification unit 134 issues a notification to prompt the user to take an action.

As described above, the user terminal apparatus according to this embodiment measures the heart rate of the user by using the blood pressure sensor and determines, based on the measured heart rate, the timing when the user should perform a predetermined action. This makes it possible to issue a notification to prompt the user to take an action.

In another embodiment, a detection unit 132 detects whether the user has entered a specific state. The specific state can be, for example, awaking. Detection may be performed based on a user input, sensor data, or information from an external device. A determination unit 133 receives heart rate data from a sensor data storage unit 131, counts the number of heartbeats (the number of times the heart beats) since wake-up of the user, and determines whether the number of heartbeats has exceeded a threshold. A threshold may be set depending on the user. For example, a threshold can be determined by a learning unit 140 based on sensor data and a user input. If the determination unit 133 determines that the number of heartbeats has exceeded the threshold, a notification unit 134 issues a notification to prompt to take an action. For example, it is believed that the mind is sharpest in a day when the number of heartbeats since awaking falls within a specific range. Accordingly, it is possible to notify the user that the current time is in a time zone during which it is preferable for the user to work or study. The number of heartbeats may be corrected based on the correlation between blood pressure values and the numbers of heartbeats. In addition, the number of heartbeats may be corrected based on the correlation between blood pressure values and the numbers of heartbeats and the correlation between blood pressure values and the amounts of exercise.

Note that the present invention is not limited to each embodiment described above, and elements can be modified and embodied in the execution stage within the spirit and scope of the invention. In addition, various inventions can be formed by proper combinations of a plurality of elements disclosed in the above embodiments. For example, several elements may be omitted from all the elements disclosed in the above embodiments. Furthermore, elements in different embodiments may be properly combined.

Although some or all of the embodiments described above can be described as in the following supplementary note as well as in the scope of claims, this is not exhaustive.

### Supplementary Note 1

A user terminal apparatus comprising:

a hardware processor; and

a memory connected to the hardware processor,

wherein the hardware processor is configured to:

detect whether a user has entered a specific state;

acquire a heart rate of the user which is measured by a sensor;

determine whether the measured heart rate is less than a threshold when it is detected that the user has entered the specific state; and

issue a notification to prompt to take an action when it is determined that the measured heart rate is less than the threshold.

1. A user terminal apparatus comprising:
a hardware processor; and
a memory connected to the hardware processor,
wherein the hardware processor is configured to:
detect whether a user has entered a specific state;
acquire a heart rate of the user which is measured by a sensor;
determine whether the measured heart rate is less than a threshold when it is detected that the user has entered the specific state; and
issue a notification to prompt to take or be administered a medicine when it is determined that the measured heart rate is less than the threshold.

2. The user terminal apparatus according to claim 1, further comprising the sensor, wherein the sensor is a blood pressure sensor configured to measure a blood pressure value per heartbeat based on the measured blood pressure waveform.

3. The user terminal apparatus according to claim 1, wherein the hardware processor is further configured to receive a user input indicating that the user has entered the specific state,
wherein the hardware processor is configured to perform detection based on the user input.

4. The user terminal apparatus according to claim 1, wherein the hardware processor is further configured to perform learning to identify that the user has entered the specific state based on sensor data obtained by the sensor,
wherein the hardware processor is configured to detect, based on sensor data obtained by the sensor, whether the user has entered the specific state.
